(19) United States
(12) Patent Application Publication
(10) Pub. No.: US 2024/0082004 A1
(43) Pub. Date: Mar. 14, 2024

(54) SENSORY MODULATION SYSTEMS FOR IMPROVING GAIT FUNCTION AND/OR BALANCE CONTROL AND RELATED METHODS

(71) Applicant: RxFunction, Inc., Eden Prairie, MN (US)

(72) Inventors: Lars Ingimar Eugen Oddsson, Edina, MN (US); Daniel Allan Samuel Nilsson, Wayzata, MN (US); Rudolf Johannes Cornelus Buijs, Lima (PE); Jennifer Lynne Johansson, Wayland, MA (US); Todd Richard Garrell, Waltham, MA (US); Benjamin Edward McDonald, Holliston, MA (US)

(21) Appl. No.: 18/464,602

(22) Filed: Sep. 11, 2023

Related U.S. Application Data
(60) Provisional application No. 63/405,115, filed on Sep. 9, 2022.

(51) Int. Cl. A61F 2/30; A61N 1/36

(52) U.S. Cl. CPC A61F 2/30721; A61N 1/36031; A61N 1/3606

(57) ABSTRACT

Systems for improvising sensorimotor function of a patient, wherein the various systems can include at least one force and/or pressure sensor associated with at least one lower limb or prosthesis of the patient, at least one motion and/or angle sensor associated with at least one lower limb or prosthesis of the patient, a processor configured to receive force and/or pressure signals and motion and/or angle signals and generate an estimated center of pressure and a center of gravity, and generate balance stimulation signals based on the estimated center of pressure and the center of gravity, and at least one sensory stimulation unit disposed on at least one lower limb or prosthesis of the patient, wherein the at least one sensory stimulation unit comprises at least two stimulators that are actuable to provide stimulation to the patient based on the balance stimulation signals.

### CROSS-REFERENCE TO RELATED APPLICATION(S)

This application claims the benefit under 35 U.S.C. § 119(e) to U.S. Provisional Application 63/405,115, filed Sep. 9, 2022 and entitled “Sensory Modulation System for Improving Balance Control,” which is hereby incorporated herein by reference in its entirety.

### FIELD

The various embodiments herein relate generally to systems for improving at least one of balance control and gait function, and more specifically to a device which measures pressure and/or force related information, and produces a sensory stimulation or notification that encodes that information.

### BACKGROUND

Loss of balance and associated falls are a significant problem for those with lower limb trauma and those who have undergone lower-limb (LL) amputations. This commonly leads to decreased activity levels, decreased participation in social activities and an increased fear of falling. In fact, 52.4% of lower extremity amputees have reported falling in the previous year and 66% of above-knee amputees report falling annually, which is twice the rate of able-bodied adults over the age of 65. Falls can have a significant impact on subsequent morbidity, disability, and mortality risk. Falls in amputees can also have serious consequences to the residual limb and damage to the prosthesis, as well as result in the lack of confidence in, and the often discontinued use of, a particular prosthesis.

Warfighters with lower limb trauma and/or loss are often young and capable of high performance at the time of their injuries. These individuals may be at increased risk of injury causing falls following rehabilitation due to their continued active lifestyle, sometimes including remaining on active duty and deployment. Even after participating in advanced rehabilitation and receiving state-of-the-art prosthetic and orthotic devices, Warfighters with lower limb trauma and/or loss are still at risk for falls.

Young and older individuals with amputation have a similar overall risk of falling. Young service members are at risk due to the more challenging activities they perform while older individuals, including individuals seen in the Veteran's Healthcare Administration (VHA) system, have greater limitations in their ability to recover. Balance impairment and associated falls are a leading concern for older individuals and individuals with amputation. Increased age increases the consequence of falls and also decreases the ability to effectively respond to a loss of balance—increasing the importance of prevention and avoiding problematic loading conditions/body positions. Impairments in vision, muscle strength, and cognition may develop with advancing age, as well as compounding medical conditions, and all contribute to an increased risk of falls in older individuals with amputation.

Common rehabilitation practices typically begin in a highly controlled environment and include basic gait and balance training activities in parallel bars to help the patient become familiar with their new sensory and motor capabilities and circumstances associated with their limb/loss or trauma. This includes “trusting” their new limb and re-learning to stand and walk. Activities gradually progress and become more difficult and can include activities specific to the requirements of the Warfighter.

Although previous research on targeted fall mitigation training has demonstrated success, these interventions are often conducted on complex, cost-prohibitive systems, such as virtual environments with perturbation platforms and treadmills that require significant space and operator training. These systems are typically not feasible for use in a typical clinical therapy setting. Also, conducting rehabilitation on a treadmill rather than in environments where sensory inputs including visual flow are more natural would be less ecologically valid. Furthermore, such systems lack important sensory stimulation, such as high fidelity extero- and proprioceptive information regarding limb loading and position that are highly relevant for gait and balance function.

There is a need in the art for an improved sensory stimulation system for improved fall mitigation and/or intervention for patients with lower limb trauma or loss as well as those with neurodegenerative diseases that cause problems with balance.

### BRIEF SUMMARY

Discussed herein are various systems, devices, and methods for improving sensorimotor function of a patient.

In Example 1, a system for improving sensorimotor function of a patient comprises at least one force and/or pressure sensor associated with at least one lower limb or prosthesis of a patient, wherein the at least one force and/or pressure sensor is configured to detect force and/or pressure information relating to the lower limb or prosthesis and transmit force and/or pressure signals based on the force and/or pressure information, at least one motion and/or angle sensor associated with at least one lower limb or prosthesis of a patient, wherein the at least one motion and/or angle sensor is configured to detect motion and/or angle information relating to the lower limb or prosthesis and transmit motion and/or angle signals based on the motion and/or angle information, a processor configured to receive the force and/or pressure signals and the motion and/or angle signals, generate a patient-specific virtual biomechanical model based on the force and/or pressure signals and the motion and/or angle signals to generate an estimated center of pressure and a center of gravity, and generate balance stimulation signals based on the estimated center of pressure and the center of gravity, and at least one sensory stimulation unit disposed on at least one lower limb or prosthesis of the patient, wherein the at least one sensory stimulation unit comprises at least two stimulators that are actuable to provide stimulation to the patient based on the balance stimulation signals.

Example 2 relates to the system according to Example 1, wherein a first of the at least one force and/or pressure sensor is associated with a first pad, wherein the first pad is disposable under a first foot or prosthetic foot of the patient.

Example 3 relates to the system according to Example 2, wherein a second of the at least one force and/or pressure sensor is associated with a second pad, wherein the second pad is disposable under a second foot or prosthetic foot of the patient.

Example 4 relates to the system according to Example 1, wherein the at least one motion and/or angle sensor comprises five motion and/or angle sensors.

Example 5 relates to the system according to Example 4, wherein each of the five motion and/or angle sensors is an inertial motion unit disposed within a sensor processing module.

Example 6 relates to the system according to Example 1, wherein the at least one sensory stimulation unit comprises a first stimulation unit disposed on a first lower limb or prosthesis of the patient and a second stimulation unit disposed on a second lower limb or prosthesis of the patient.

Example 7 relates to the system according to Example 1, wherein the at least one sensory stimulation unit comprises four stimulators.

Example 8 relates to the system according to Example 1, further comprising a user interface operably coupled to the processor, wherein the user interface is configured to display the patient-specific virtual biomechanical model.

Example 9 relates to the system according to Example 8, wherein the user interface comprises an application in a mobile device.

Example 10 relates to the system according to Example 9, wherein the mobile device comprises a laptop or a smartphone.

In Example 11, a system for improving sensorimotor function of a patient comprises at least one force and/or pressure sensor associated with at least one lower limb or prosthesis of a patient, wherein the at least one force and/or pressure sensor is configured to detect force and/or pressure information relating to the lower limb or prosthesis and transmit force and/or pressure signals based on the force and/or pressure information, at least one motion and/or angle sensor associated with at least one lower limb or prosthesis of a patient, wherein the at least one motion and/or angle sensor is configured to detect motion and/or angle information relating to the lower limb or prosthesis and transmit motion and/or angle signals based on the motion and/or angle information, a processor configured to receive the force and/or pressure signals and the motion and/or angle signals, generate a patient-specific virtual biomechanical model based on the force and/or pressure signals and the motion and/or angle signals to generate an estimated center of pressure and a center of gravity, and generate balance stimulation signals based on the estimated center of pressure and the center of gravity, at least one sensory stimulation unit disposed on at least one lower limb or prosthesis of the patient, wherein the at least one sensory stimulation unit comprises at least two stimulators that are actuable to provide stimulation to the patient based on the balance stimulation signals, and a user interface operably coupled to the processor, wherein the user interface is configured to receive information from the processor about the patient-specific virtual biomechanical model and display the patient-specific virtual biomechanical model based on the information from the processor.

Example 12 relates to the system according to Example 11, wherein a first of the at least one force and/or pressure sensor is associated with a first pad, wherein the first pad is disposable under a first foot or prosthetic foot of the patient and a second of the at least one force and/or pressure sensor is associated with a second pad, wherein the second pad is disposable under a second foot or prosthetic foot of the patient.

Example 13 relates to the system according to Example 11, wherein the at least one motion and/or angle sensor comprises five motion and/or angle sensors, wherein first and second motion and/or angle sensors are disposed on a first lower limb or prosthesis of the patient, third and fourth motion and/or angle sensors are disposed on a second lower limb or prosthesis of the patient, and a fifth motion and/or angle sensor is disposed on a lower back of the patient.

Example 14 relates to the system according to Example 13, wherein each of the five motion and/or angle sensors is an inertial motion unit disposed within a sensor processing module, wherein the fifth motion and/or angle sensor is operably coupled to a local central processor, wherein the local central processor is in communication with the processor.

Example 15 relates to the system according to Example 11, wherein the at least one sensory stimulation unit comprises a first stimulation unit disposed on a first lower limb or prosthesis of the patient and a second stimulation unit disposed on a second lower limb or prosthesis of the patient, wherein each of the first and second stimulation units comprises a band configured to be couplable to a lower limb or prosthesis, the at least two stimulators comprising four stimulators attached to the band, and one of the at least one motion and/or angle sensor associated with one of the four stimulators.

Example 16 relates to the system according to Example 11, wherein the user interface comprises an application in a mobile device, wherein the mobile device comprises a laptop, or a smartphone.

In Example 17, a system for improving sensorimotor function of a patient comprises a first footpad unit comprising a first footpad comprising at least one first force and/or pressure sensor positionable under a first foot or prosthetic foot of a first lower limb or prosthesis of a patient, and a second footpad unit comprising a second footpad comprising at least one second force and/or pressure sensor positionable under a second foot or prosthetic foot of a second lower limb or prosthesis of the patient, wherein each of the at least one first and second force and/or pressure sensors are configured to detect force and/or pressure information relating to the first and second lower limbs or prostheses, respectively, and transmit force and/or pressure signals based on the force and/or pressure information. The system further comprises first and second sensor processing modules comprising at least one first motion and/or angle sensor associated with the first lower limb or prosthesis of the patient, third and fourth sensor processing modules comprising at least one second motion and/or angle sensor associated with the second lower limb or prosthesis of the patient, and a fifth sensor processing module comprising at least one third motion and/or angle sensor associated with a lower back of the patient, wherein each of the at least one first, second, and third motion and/or angle sensors is configured to detect motion and/or angle information and transmit motion and/or angle signals based on the motion and/or angle information. The system also comprises a processor configured to receive the force and/or pressure signals and the motion and/or angle signals, generate a patient-specific virtual biomechanical model based on the force and/or pressure signals and the motion and/or angle signals to generate an estimated center of pressure and a center of gravity, and generate balance stimulation signals based on the estimated center of pressure and the center of gravity. In addition, the system comprises at least one sensory stimulation unit disposed on at least one lower limb or prosthesis of the patient, wherein the at least one sensory stimulation unit comprises at least two stimulators that are actuable to provide stimulation to the patient based on the balance stimulation signals. And the system comprises a user interface operably coupled to the processor, wherein the user interface is configured to receive information from the processor about the patient-specific virtual biomechanical model and display the patient-specific virtual biomechanical model based on the information from the processor.

Example 18 relates to the system according to Example 17, wherein the fifth sensor processing module comprises a local central processor, wherein the local central processor is in communication with the processor.

Example 19 relates to the system according to Example 17, wherein the at least one sensory stimulation unit comprises first and second stimulation units. The first stimulation unit is disposed on the first lower limb or prosthesis of the patient and comprises a first band configured to be couplable to the first lower limb or prosthesis, four first stimulators attached to the first band, and one of the first and second sensor processing modules associated with one of the four stimulators. The second stimulation unit is disposed on the second lower limb or prosthesis of the patient and comprises a second band configured to be couplable to the second lower limb or prosthesis, four second stimulators attached to the second band, and one of the third and fourth sensor processing modules associated with one of the four stimulators.

Example 20 relates to the system according to Example 17, wherein the user interface comprises an application in a mobile device, wherein the mobile device comprises a laptop or a smartphone.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a schematic depiction of a system for improving sensorimotor function of a patient, according to one embodiment.

FIG. 2A is a perspective view of a footpad unit, according to one embodiment.

FIG. 2B is a perspective view of a haptic stimulation unit, according to one embodiment.

FIG. 3A is a schematic depiction of a standing patient wearing a set of sensory processing modules, according to one embodiment.

FIG. 3B is a reproductive image of an electronically-generated human model replicating the motion and position of the limbs of the patient in FIG. 3A, according to one embodiment.

FIG. 4A is a schematic depiction of the patient of FIG. 3A in which the patient has placed her right leg forward while walking, according to one embodiment.

FIG. 4B is a reproductive image of an electronically-generated human model replicating the motion and position of the limbs of the patient in FIG. 4A, according to one embodiment.

FIG. 5A is a schematic depiction of the patient of FIG. 3A in which the patient has raised her right foot while walking, according to one embodiment.

FIG. 5B is a reproductive image of an electronically-generated human model replicating the motion and position of the limbs of the patient in FIG. 5A, according to one embodiment.

FIG. 6A is a back view of a patient wearing force/pressure sensors and motion/angle sensors that has placed his right leg forward while walking with schematic depictions of the calculation of the center of pressure and the center of mass of the patient, according to one embodiment.

FIG. 6B is a back view of the patient of FIG. 6A when the patient has placed his right foot on the ground while walking with schematic depictions of the calculations of the center of pressure and the center of mass of the patient, according to one embodiment.

FIG. 6C is a back view of the patient of FIG. 6A when the system if providing stimulation to the patient based on the calculations of the center of pressure and the center of mass, according to one embodiment.

FIG. 7 is a flow chart depicting the steps of a method of tracking the movement/activity of a patient and providing stimulation to the patient based on those movements/activity, according to one embodiment.

FIGS. 8A and 8B are representative depictions of the interface of a mobile device, according to one embodiment.

FIG. 9 is a schematic depiction of a computing device for use or combination with any of the systems disclosed or contemplated herein, according to one embodiment.

### DETAILED DESCRIPTION

The various embodiments herein relate to systems and devices for providing relevant sensory stimulation to a patient. More specifically, the various systems and devices herein have both force/pressure sensors and motion/angle sensors which provide information to a processor, which in turn uses the information to provide real-time sensory stimulation to the patient relating to the patient's balance and/or loss of balance. Additional system and device embodiments can include a patient-specific virtual model created by the system processor and/or system software to assimilate the various force/pressure/motion/angle and other balance parameters for purposes of generating refined and precise sensory stimulation to the patient. Certain implementations can be used for fall mitigation training by a patient with lower limb trauma or loss, including as part of the rehabilitation care. That is, some of the various system and device embodiments disclosed or contemplated herein can facilitate and/or enhance sensorimotor function of a patient with lower limb trauma or loss, while other embodiments can be used to facilitate/enhance the sensorimotor function of patients with other conditions, including, for example, neurological conditions such as stroke. Further, in certain exemplary implementations, the various systems and devices herein can facilitate and/or enhance sensorimotor function of a patient in any environment and with any of the conditions disclosed or contemplated herein.

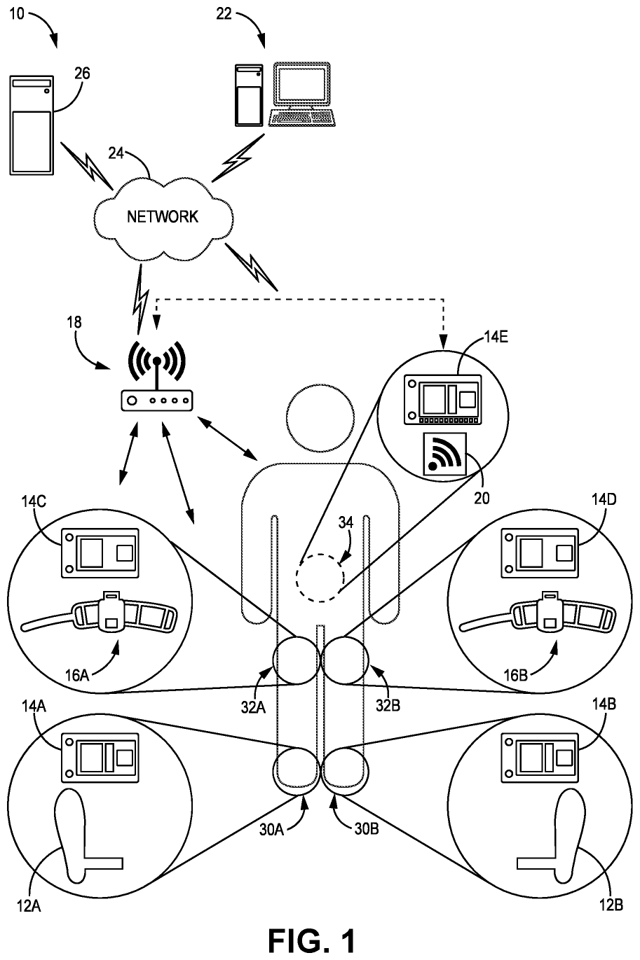

One exemplary system 10, according to one embodiment, is depicted schematically in FIG. 1. This system 10 has two footpad units 12A, 12B, with the right footpad unit 12A having a footpad positionable under the patient's right foot (or artificial limb) 30A and the left footpad unit 12B having a footpad positionable under the patient's left foot (or artificial limb) 30B, with each of those footpads of the footpad units 12A, 12B having at least one force or pressure sensor. Further, the system 10 also has five motion and angle sensors 14A, 14B, 14C, 14D, 14E, with the right foot sensor 14A attached to or disposed near the right foot (or artificial limb) 30A, the left foot sensor 14B attached to or disposed near the left foot (or artificial limb) 30B, the right leg sensor 14C attached to or disposed near the right thigh (or artificial limb) 32A, the left leg sensor 14D attached to or disposed near the left thigh (or artificial limb) 32B, and the lower back sensor 14E attached to or disposed near the lower lumbar region 34 of the patient. In addition, the system 10 has two haptic sensory stimulation units 16A, 16B, with the right leg stimulation unit 16A attached to the right thigh 32A and the left leg stimulation unit 16B attached to the left thigh 32B. In accordance with certain implementations, the right and left leg sensors 14C, 14D can be incorporated into the sensory stimulation units 16A, 16B and the right and left foot sensors 14A, 14B can be incorporated into the right and left footpad units 12A, 12B, as will be discussed in additional detail below. Applicants note that, due to the use of the various device/system embodiments herein by patients with damaged limbs, the use of the term foot or limb herein can also refer to any type of prosthesis or artificial limb as well. Further, the terms “artificial limb” and “prothesis” are intended to have the same meaning and be interchangeable as used herein.

The system 10 also has a central processor 18 that is wirelessly coupled to the force/pressure sensors of the footpad units 12A-12B, the motion/angle sensors 14A-14E, and the haptic stimulation units 16A, 16B such that the information from the force/pressure sensors of the units 12A-12B and the motion/angle sensors 14A-14E can be transmitted or otherwise communicated to the central processor 18 and the processor 18 can process the information and transmit sensory stimulation instructions to the sensory stimulation units 16A, 16B, thereby providing sensory stimulation to the patient as described in additional detail below. That is, the central processor 18 uses the information from the sensors of the units 12A-B and the sensors 14A-E to make calculations regarding when to activate the stimulation units 16A, 16B to provide sensory stimulation to the patient during use, as will be discussed in further detail below. In one implementation, the stimulation units 16A, 16B provide sensory tactile stimulation in the form of vibrations. Alternatively, the stimulation units 16A, 16B can provide any form of sensory stimulation.

In alternative embodiments, the system can have one sensory stimulation unit (including, for example, in situations in which one of the two limbs has been amputated or severely damaged). In further alternatives, three or more stimulation units can be used. In accordance with other alternative implementations, the number of motion/angle sensors can be one, two, three, four, six, seven, eight, nine, ten, or any other number of sensors that can be positioned strategically on the patient to collect information. According to additional alternatives, each of the sensors 14A-14E can have local processors associated with the sensors 14A-14E such that each can perform local processing of the information collected by its respective sensor 14A-14E.

In one specific alternative embodiment as shown in FIG. 1, the lower back sensor 14E has a local central processor 20 coupled thereto that is in wireless communication with the force/pressure sensors in the footpad units 12A, 12B and the other motion/angle sensors 14A-14D such that the local central processor 20 performs the central processing operations described above and communicates with the central processor 18 to transmit data and other information as will be described in additional detail below.

In addition, the system 10 can also have at least one computer or mobile device 22 that is coupled to the processor 18 and/or the processor 20 via a network 24 such as a local area network or the internet 24. Further, one or more servers 26 can also be coupled to the system 10—either directly to the computer/mobile device 22 or through the network 24—such that the servers 26 can perform any of the processing disclosed or contemplated herein. As will be discussed elsewhere herein, the computer or mobile device 22 can be used by a clinician to set the parameters for the use of the system 10 (or any system embodiment herein) as will be described in additional detail herein and/or to receive results relating to the use of the system by a patient. Alternatively, the computer or mobile device 22 can be used by a patient during use of the system 10, to input information into the system 10, and/or to access information about the patient's use of the system 10 or analysis of such use generated by the system 10, as will be described in further detail below. For example, in one embodiment as will be described in additional detail below, an application can be loaded onto the patient's phone such that the phone operates as a mobile device 22 that can be used to interface with the system 10 in the ways described above and elsewhere herein.

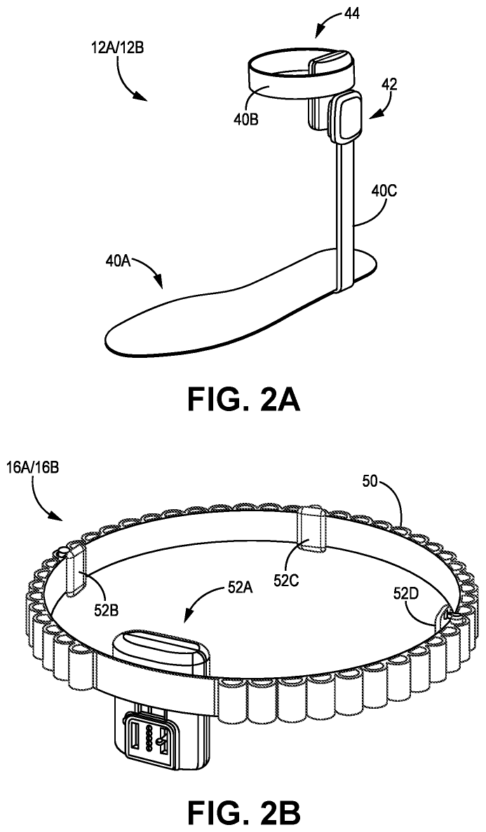

One exemplary embodiment of a footpad unit 12A/12B is depicted in FIG. 2A. As noted above, each footpad unit 12A, 12B has a footpad 40A for placement under the foot of the patient that has at least one force/pressure sensor (not shown) disposed within/integral with the footpad 40A to sense force and/or pressure created by force of the patient's foot contacting the footpad 40A. More specifically, in one specific embodiment, each footpad 40A of each footpad unit 12A, 12B has four force/pressure sensors (not shown): a front sensor, a back sensor, an outer sensor, and an inner sensor. Alternatively, each footpad 40A can have two, three, five, six, seven, eight, nine, ten, eleven, twelve, or any number of sensors as needed to track the center of pressure (“COP”) relating to each foot of the patient. For example, in certain embodiments, the footpads 40A and/or the entire footpad units 12A, 12B can be the commercially-available footpads or units available as part of the Walkasins® system. According to another implementation, the footpads 40A and/or the units 12A, 12B can be any of the footpad or footpad unit embodiments as disclosed in U.S. Pat. No. 8,974,402, issued on Mar. 10, 2015 and entitled “Sensor Prosthetic for Improved Balance Control,” which is hereby incorporated herein by reference in its entirety. Each unit 12A/12B also has a leg band 40B that can be positioned around and attached to the lower leg of the patient and a connector band 40C that couples the band 40B to the footpad 40A. In the specific implementation as shown, each footpad unit 12A/12B has a local processor 42 coupled to the connector band 40C (or the leg band 40B), such that the local processor 42 can receive the signals from the pressure/force sensors in the footpad 40A, process the signals, and transmit information relating to those signals and the processing to the central processor (such as central processor 18 and/or local central processor 20 as discussed above). Further, each unit 12A/12B can also have one of the motion and angle sensors 44 coupled to the leg band 40B (or the connector band 40C) as well. For example, the motion and angle sensor 44 in the right unit 12A can be the sensor 14A discussed above, while the motion and angle sensor 44 in the left unit 12B can be the sensor 14B discussed above.

In certain embodiments, each footpad (such as footpads 40A in footpad units 12A, 12B) can provide foot pressure data used to calculate the center of pressure (“COP”). That is, an exemplary footpad contains pressure sensors positioned at locations corresponding to the anatomical pressure distribution of the plantar surface of the foot. The back sensor covers most of the pressure from the heel of the foot. The outer sensor covers the lateral side of the foot up to the fifth metatarsal heads. The front sensor is located at the ball of the foot between the first and fifth metatarsal heads. The inner sensor is located medial side of the foot up to the first metatarsal head such that the sensor is loaded by both the first metatarsal head and by the surface under the arch. Regardless of the number of pressure sensors and the positioning thereof, the foot pressure amplitude and distribution data from these sensors can be used to estimate the COP of the patient during activities such as standing and walking and the like.

An exemplary implementation of a haptic stimulation unit 16A/16B is depicted in FIG. 2B. The stimulation unit 16A/16B in this embodiment has a band 50 with four vibrotactile actuators 52A, 52B, 52C, 52D disposed strategically around the band 50 such that they are positioned in the anterior, posterior, medial and lateral positions with respect to the patient's leg. In certain embodiments, the haptic stimulation unit 16A/16B can be the commercially available stimulation units available as part of the Walkasins® system, or alternatively, can be any of the stimulation units disclosed in U.S. Pat. No. 8,974,402, which is incorporated by reference above. Further, one of the actuators 52A as shown can also be a motion and angle sensor 52A coupled to the band 50 as well. For example, the actuator 52A in the right haptic stimulation unit 16A can also be the motion and angle sensor 14C discussed above, while the actuator 52A in the left haptic stimulation unit 16B can also be the motion and angle sensor 14D discussed above.

According to some implementations, each motion/angle sensor is an inertial motion unit (“IMU”). Exemplary commercially-available IMUs include ST Micro ISM330 and Invensense/TDK ICM-20948. Further, in certain embodiments as noted above, each of the motion/angle sensors (such as sensors 14A-14E) in the system 10 is incorporated into a unit that includes a local processor. Such a unit can be referred to herein as a sensor processing module (“SPM”) such that the motion and angle sensors 14A-14E are also SPMs 14A-14E. The SPM can capture IMU data, and provide local sensor fusion functionality and connectivity. One exemplary SPM embodiment has an IMU (such as a 9-axis, e.g. Bosch BN0055), a microprocessor (such as ARM Cortex-M4 with floating point hardware or similar), a wireless transceiver (such as Bluetooth Low Energy 5.0+), a battery (such as Lithium-Polymer or other high current output/low volume battery required for motor activation), an analog input port (with amplification and filtering circuitry to read resistances from the foot sensors), a vibrotactile actuator (such as a Linear Resonant Actuator), and drive circuitry to couple to the additional vibrotactile actuators in the haptic stimulation unit, or any combination of these components/features. Further, certain SPM embodiments will be capable of wireless over-the-air (OTA) updates and configuration to firmware, fusing sensor information and transferring relevant data to other system modules, processing model data and providing activation signals to other SPMs, providing power to the SPM itself as well as to connected peripherals, providing an amplitude and frequency modulated signal to drive actuators in the haptic stimulation unit, and reading analog signals on at least 4 input channels, or any combination of these capabilities.

In certain embodiments, the central processor (such as central processor 18) can calculate patient-specific biomechanical model data (including an estimation of the center of mass (“COM”) of the patient), control the system configuration, provide secure storage, and analyze data, or any combination thereof. Data collected to build the patient-specific model (as discussed in further detail below) can be locally logged and analyzed by the central processor. Further, in various aspects, the central processor can additionally perform as a gateway to connect the system 10 to remote hardware (such as the server 26 and/or the computer/mobile device 22 discussed above) for analysis or download of data for storage. In some implementations, an application running on an off-the-shelf mobile device (such as device 22) such as a phone, tablet, or laptop can provide the central processor functionality. That is, the central processor 18 can be wirelessly connected to the mobile device (such as device 22) such that the mobile device and central processor can communicate. In certain embodiments, the central processor 18 can provide the functionality of both a gateway as well as a control interface for the clinician/technician or patient during the sensory stimulation exercises. In exemplary implementation, the central processor 18 can communicate with the SPMs 14A-14E to retrieve and forward data for secure storage, display relevant use data or live streaming data from the system, calculate the patient-specific model (in real-time or offline), send stimulation activation parameters to the system 10, manage, configurate and calibrate the SPMs 14A-14E, or any combination of these actions. In specific examples, the central processor 18 can be a laptop or a mobile phone such as a Samsung galaxy S9.

In various embodiments, communication between the central processor 18 and the SPMs 14A-14E, between the SPMs 14A-14E, and/or between the servers, computer/mobile device 22, the processor 18, and/or the processor 20 can be communication via physical connections (wires) or via wireless communication. For example, the wireless communication can be BLE 5.0 (Bluetooth Low Energy). Alternatively, other known wireless technologies, such as ANT, Thread, Zigbee, Wi-Fi or proprietary protocols in the ISM bands could be used.

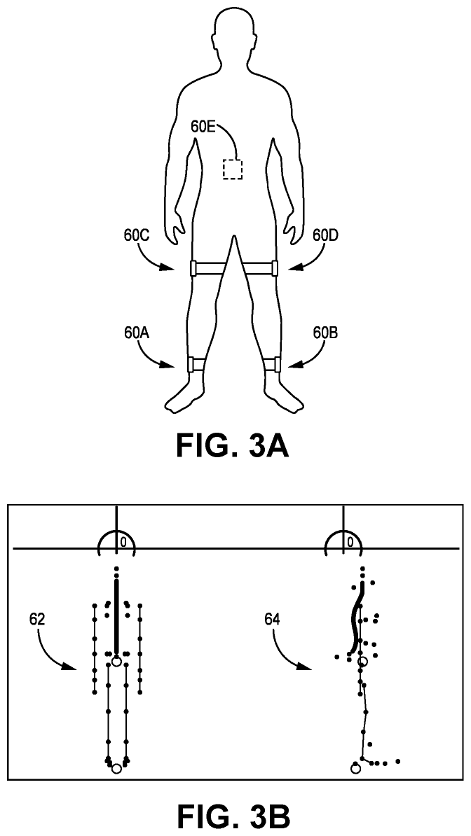

As mentioned above, in certain implementations, the system 10 can use an electronic full-body human model, with an exemplary version depicted in FIGS. 3B, 4B, and 5B (and discussed in further detail below). In certain embodiments, the system embodiments herein can utilize sensor information and generate the model in real-time. The model is generated by the information from the motion/angle sensors (such as the sensors 14A-14E discussed above with respect to the system 10) attached to the patient. The parameters that can be tracked by the motion/angle sensors can include, but are not limited to, heel strike (“HS”) and toe off (“TO”) accuracy, step length, step width, toe clearance during swing, thigh position, anteroposterior (“A/P”) and mediolateral (“M/L”) angular momentum, etc. In one embodiment, the model is written in Java, but can be created with any software.

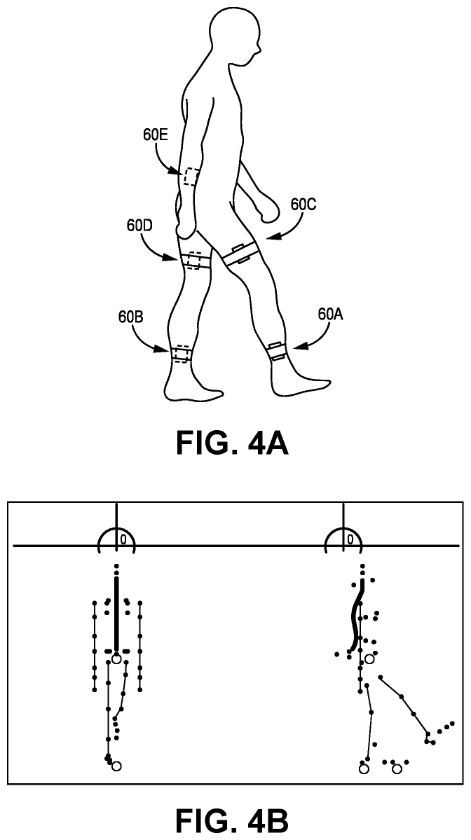

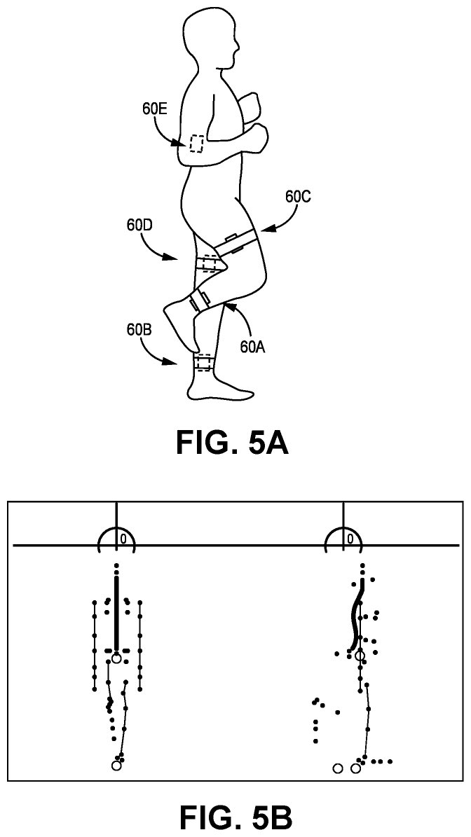

One example of IMUs being used to generate an electronic, real-time full-body human model is shown in FIGS. 3A-5B. More specifically, as shown in FIG. 3A, the IMUs 60A, 60B, 60C, 60D, 60E are positioned on the patient in order to track the movement of the patient. More specifically, the IMU 60A is attached to the patient's right foot or ankle, the IMU 60B is attached to the patient's left foot or ankle, the IMU 60C is attached to the patient's right thigh, the IMU 60D is attached to the patient's left thigh, and the IMU 60E is attached to the patient's lower back in a fashion similar to that described above with respect to FIG. 1. Thus, the resulting model generated by the IMUs 60A-60E (as positioned in FIG. 3A) is shown in FIG. 3B, which depicts a graphic user interface displaying the human model, including a front view 62 and a side view 64. According to one embodiment, the views 62, 64 of the model can be displayed on a computer, tablet, or mobile device (such as device 22) as discussed elsewhere herein. In FIG. 4A, the patient is walking such that the right leg moves forward in a hip flexion movement with the right leg sensors 60A, 60C tracking that movement such that it is reflected by the movement of the model in FIG. 4B. Further, in FIG. 5A, the patient's right leg moves into a right knee flexion movement with the right leg sensors 60A, 60B tracking that movement such that it is reflected in the movement of the model in FIG. 5B. Thus, the various sensors 60A-60E make it possible to track all of the standing, walking, and/or running movements of a patient such that the movements can be reflected in the movement of the electronic model in a similar fashion as above.

In various embodiments, the virtual biomechanical model can be “fitted” to the specific patient using the system (such as system 10) by registering certain basic anthropometrical parameters of interest with the system, including, but not limited to, subject height, subject weight, sex, etc.

As a result, the various system embodiments herein (such as system 10 above) can be used for various use cases relating to improvement of sensorimotor function (including, in some exemplary cases, for rehabilitation) via sensory stimulation of a patient with lower limb trauma, lower limb loss, or other malfunction such as stroke or other neurological condition or disease.

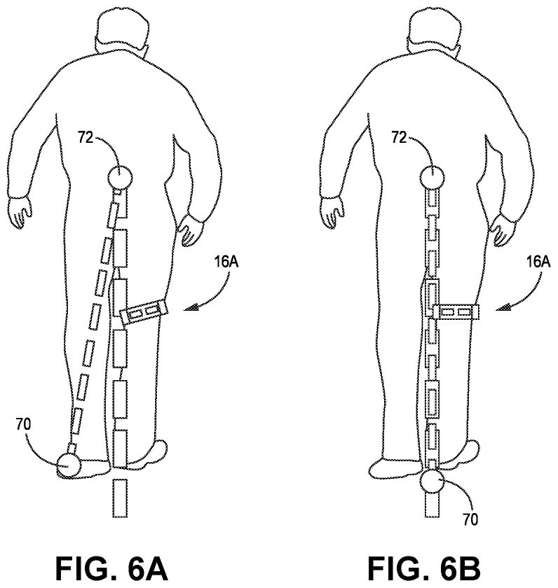

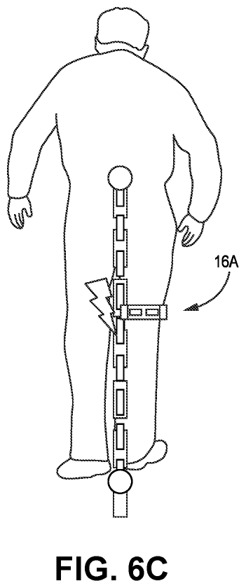

For example, in one embodiment as shown in FIGS. 6A-6C, a walking patient can utilize a system (such as system 10) according to the embodiments herein in the following fashion. The patient can wear a set of sensors similar to the sensors 12A-12B, 14A-14E in system 10 above. As shown in FIG. 6A, during use by the patient, the system (such as system 10) can track and calculate the center of pressure 70 and the center of mass 72 of the patient as shown. Further, while the patient is walking as shown in FIG. 6B, the patient takes a step with his right foot that is too narrow such that the footpad 12A and the sensors 14A, 14B associated with the right foot detects a right heel strike that may be too medial. Thus, the system transmit signals in real-time to the right haptic stimulation unit 16A that indicates to the patient's nervous system about the narrow step as shown in FIG. 6C.

Further, the various system embodiments herein (such as system 10) can be used to monitor and provide sensory stimuli relating to various activities including, but not limited to, the following: static standing weight bearing with vibrational gradient for intensity, step length for better symmetry, balance during weight shifting, and terminal stance toe load and/or timing during gait. In further implementations, the system can be used to monitor and provide stimulation for various other physical activities involving the lower limbs and postural control and/or balance.

In accordance with a further embodiment, the system can have various exemplary sensory stimulation modes that can be used to treat various patients. Table 1 below provides an exemplary, non-exhaustive list of such therapy modes.

TABLE 1

| | STIMULATION | INPUT | | TYPE OF |
|---|---|---|---|---|
| CATEGORY | MODALITY | PARAMETER(S) | SENSOR | STIMULATION |
| Standing | Weight shifting - | Total load on each | Right and Left | Pulsing stimuli |
| Activities (SA) | basic | foot (x sensors/foot) | Total Foot Pressure | (load on each |
| | (mediolateral) | | | foot maps to a |
| | | | | frequency on |
| | | | | same side of |
| | | | | trunk) |
| | | | | Single stimulus |
| | | | | Visual |
| Standing | Weight shifting - | Total load for each | Right and Left Foot | Pulsing |
| Activities (SA) | advanced | foot | Pressure | stimulations |
| | | CoM | Model | |
| | | CoP | Model | |
| | | Stance width | Model | Visual |
| | | Subject's | Clinician input | Single stimulus |
| | | anthropometric | | |
| | | measurements | | |
| | | Leg angles | Model | |
| Gait Activities | Toe load target for | Force | Right and Left | Single stimulus |
| (GA) AP | terminal stance | | Pressure insole | (or pulse flow) |
| | | | (toe sensor) | |
| | | Time | Stance and swing | |
| | | | phase measurements | |
| | | Shank Angle (relative | Shank IMU | |
| | | to vertical) | | |
| | Heel strike/toe off | Force | Heel sensor | Single stimulus |
| | indication | Force | Toe sensor | (“flow” of |
| | | | | stimuli from |
| | | | | heel to toe) |
| | Axial trunk | Trunk sensor rotation | Lumbar IMU | Single stimulus |
| | rotation - basic | | | (stimulation |
| | | | | “flow”) |
| Gait Activities | Step symmetry - ML | CoM | Swing plane | Single stimulus |
| (GA) ML | advanced | | deviation, leg | |
| | | | IMU/s | |
| | | CoP | ML Heelstrike | |
| | | | location with | |
| | | | respect to COM | |

| | ACTUATOR | WHEN TO PROVIDE | |
|---|---|---|---|
| CATEGORY | LOCATION | STIMULATION | NOTES |
| Standing | Trunk in | 50/50 weight | Two separate settings |
| Activities (SA) | mediolateral | distribution (+/− error | (balanced 50/50 or clinician |
| | direction they | range. More frequent | set load threshold). |
| | are leaning in. | pulsing the closer | Have clinician instruct to |
| | | (positive mode) or | stand straight and not bend |
| | | furthre (negative | at the hip/waist. |
| | | mode) they get from | |
| | | “balanced” range. | |
| | Trunk in direction | Positive mode: When | |
| | they are leaning | clinician set | |
| | in. | threshold is reached | |
| | N/A | Visual stimuli: bars | |
| | | showing % weight | |
| | | distribution for each | |
| | | foot (to view during | |
| | | above modes) | |
| Standing | Trunk in direction | More frequent | CoM projection on CoP |
| Activities (SA) | they are leaning | pulsing the closer | Exploring base of support |
| | in. | (positive mode) or | and stability. |
| | | furthre (negative | If incorporating CoM is too |
| | | mode) they get from | slow, just use CoP |
| | | the clinician set | Shows off system in a less |
| | | thresholds. | time critical sitaution than |
| | N/A | Bullseye location on | gait. |
| | Trunk AP and ML | screen for patients to | |
| | | reach. | |
| Gait Activities | Trunk (on the | Positive Mode: When | Time of gait cycle may be |
| (GA) AP | side of toe load | force and/or time | calculated with previous |
| | detection). | threshold is reached. | steps to undestand timing |
| | If sound side, | | parameter. |
| | can stimulate on | | Thresholds and which foot |
| | leg near the foot. | | to detect toe load can be |
| | | | modified by clinician. |
| | | | Give ability for negative |
| | | | mode (when threshold/s |
| | | | have not been reached) |
| | [symbol] side of HS/TO | Positive Stimulation: | Ability for the clinician to |
| | detection) 45 deg | When force | choose only one parameter |
| | rotation of belt | threshold is reached | (heel strike OR toe off) |
| | when using both | | |
| | sides. | | |
| | If sound side, | | |
| | can stimulate on | | |
| | leg near the foot. | | |
| | Trunk (in direction | When trunk sensor | |
| | of rotation) | rotation threshold | |
| | | has been reached. | |
| Gait Activities | Trunk in direction | When inclination | Focuses on inclination angle |
| (GA) ML | of imbalance/leaning | angle crosses | (CoM projectionon CoP). |
| | | threshold. | Set a threshold for when a |
| | | | step is likely to cause an |
| | | | imbalance. |
| | | | Shows off the system |
| | | | because uses the model in a |
| | | | time critical situation. |

[symbol]

indicates data missing or illegible when filed

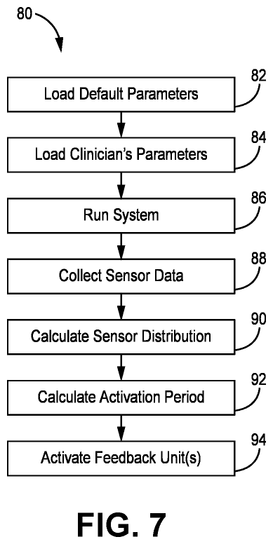

For example, according to one exemplary embodiment as shown in FIG. 7, the system (such as system 10) can be used to perform a method of tracking weight shifting of a patient and provide stimulation regarding same 80. Specifically, the therapy mode in this particular implementation can be “Weight shifting—basic” as set forth in the first row of Table 1 above.

In certain embodiments, the first step of the method 80 is to enter the default parameters into the system (block 82). Such parameters can include, for example, the duration of the stimulation, the target weight distribution, the type of stimuli (continuous vs. repeating, etc.), and/or whether the stimulation is positive or negative, among other potential parameters. In one embodiment, the default parameters are entered (block 82) via an application on a mobile device (such as device 22 as discussed above) by a clinician. Alternatively, the default parameters can be entered by a system administrator or other individual. In a further implementation, the default parameters are entered via any known interface when the system (such as system 10) is first set up by the health care facility, clinician, or patient. In yet another alternative, the default parameters are built into the system.

Once the default parameters are entered, or as a first step in those embodiments in which the default parameters have previously been entered, the clinician can then enter the clinician's preferred parameters (block 84). Such parameters can include, for example, any of the default parameters discussed above. As such, the clinician (or any other user) can incorporate her own preferred parameters for a specific patient or use that overrides the existing default parameters. Alternatively, the clinician or other user can opt to use the default parameters (and thus not enter any new/different parameters).

Once the preferred parameters have been established, the next step is to begin operating the system by attaching the sensors/devices to a patient and tracking the patient's movement/activities (block 86). In this specific embodiment, the patient is standing and any weight shifting between the patient's two legs is tracked, as noted above.

Once the system is activated, the data from the sensors (including, for example, the sensors in the footpads—such as footpads in the footpad units 12A, 12B—and/or the motion and angle sensors—such as sensors 14A-14E) is collected (block 88). For example, in one implementation in which the lower back sensor (such as sensor 14E) includes a local processor (such as processor 20), the footpad sensors (such as in the footpad units 12A, 12B) collect force and/or pressure data and transmit it to the lower back sensor (such as sensor 14E). That is, the sensors in the right and left footpads (such as the footpads in units 12A, 12B) track the amount of force applied thereto based on the stance of the patient. If the patient shifts her weight from one foot to the other, then the weight distribution shifts accordingly, and the sensors in the footpad units 12A, 12B track that shift and transmit that data to the local processor 20. At this point, the local processor (such as processor 20) can process the information and perform the calculations as discussed below relating to this method 80. Alternatively, the lower back sensor 14E and/or local processor 20 can transmit the data to the central processor 18 such that the central processor 18 can process the information and perform the calculations.

At this point, the collected data is used to calculate the weight distribution and thus the center of gravity of the patient based on the sensor data (block 90). That is, the data from the sensors in the right and left footpads (such as footpads of units 12A, 12B) is collected, combined, and processed by the local processor 20 (and/or the central processor 18) to calculate the patient's center of gravity at any given time.

Once the patient's weight distribution/center of gravity is calculated, that data is compared to the target weight distribution/center of gravity to identify the difference therebetween (if any) and calculate the stimulation unit activation period based on same (block 92). That is, the difference between the actual weight distribution and the target distribution is first calculated. As a result, the data can be used to track any shift in the center of gravity, including any shift away from a target weight distribution or center of gravity location. That is, any movement of the patient's weight distribution away from or toward a desired weight distribution/center of gravity can be calculated based on the collected data and the preset target weight distribution/center of gravity. Once the difference calculated, that information is used to determine the activation period of the stimulation unit. In other words, the amount of the difference determines the activation period. For example, the greater the difference, then the farther that the actual center of gravity is from the target center of gravity (the more that the patient has shifted her weight away from the target center of gravity). And the activation period of the stimulation unit is dependent on the distance between the actual center of gravity and the target center of gravity. For example, in one embodiment, the greater the distance, the greater the activation period (and thus the longer the duration and/or the greater the intensity of the stimulus provided to the patient at the stimulation unit). Alternatively, the greater the distance, the shorter the activation period (and thus the greater the number of activations—vibrations, beeps, or the like—over a shorter period of time provided to the patient at the stimulation unit). In any of the embodiments herein, the parameters provided by the system (the default parameters) or the parameters provided by the clinician or other user as described above will be used as part of the calculation to determine the activation period.

In one specific exemplary implementation, a predetermine threshold of movement is set in the parameters such that when the patient shifts her weight to one leg or the other by a sufficient amount to cross that threshold, then the calculation triggers activation. In such an embodiment, a target weight distribution/center of gravity range can be set such that the system does not cause the activation of the stimulation units so long as the patient remains within that target range (a “balance deadzone”). Thus, calculations cause activation of the stimulation units only when the threshold beyond the target range is hit and/or surpassed.

Further, the calculation can also be used to determine which of the two stimulation units 16A, 16B is activated to provide sensory stimulation. That is, according to the preset parameters, the stimulation unit 16A/16B on the leg to which more of the patient's weight has been shifted can be activated to provide stimulation. Alternatively, the preset parameters can be set such that the unit 16A/16B on the leg to which less of the weight has been shifted can be activated.

Once the activation period has been calculated, that calculation is used to transmit appropriate signals from the local processor 20 (or the central processor 18) to activate the stimulation units 16A, 16B (or the appropriate unit 16A/16B) according to the parameters and as determined by the calculations as discussed above (block 94).

Alternatively, the same or a similar process can be used to perform any of the sensory stimulation modes listed in Table 1 above or any other sensory stimulation mode disclosed or contemplated herein. Further, it is noted that if a certain sensory stimulation mode (such as any of those set forth in Table 1, for example) only uses a subset of the various system components as disclosed or contemplated herein, only those components to be utilized would need to be incorporated into the physical system and worn by the patient.

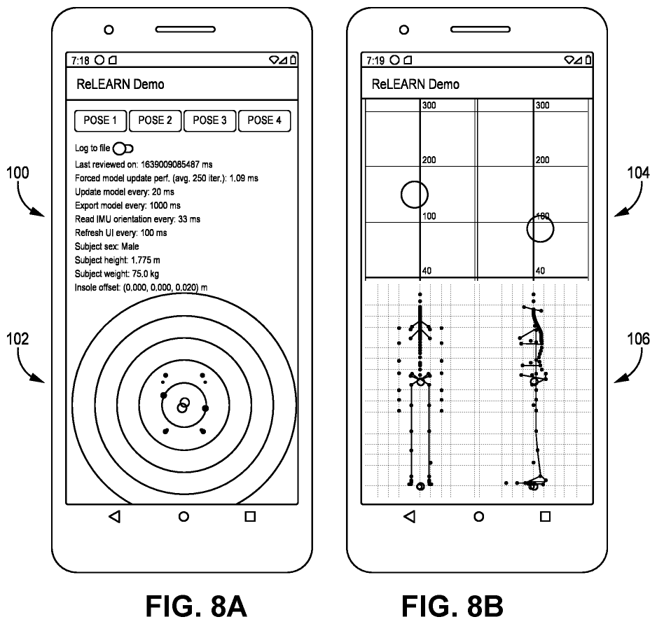

In accordance with certain implementations, the system (such as system 10) can operate in conjunction with a mobile device such as a smartphone (such as device 22). For example, as shown in FIGS. 8A and 8B, an application is provided for a smartphone with a user interface that can display the human model (as best shown in FIG. 8B) in a fashion similar to the graphic user interface discussed in further detail above and depicted in FIGS. 3B, 4B, and 5B. The smartphone (such as mobile device 22) can communicate wirelessly with the system (such as system 10) by communicating with the central processor (such as processor 18) and/or the local central processor (such as processor 20).

FIG. 8A depicts the application display in which the timing characteristics and other details about the patient are provided at the top of the screen 100, while the bottom portion of the screen 102 displays a real-time top view of the following points of interest: front and back of both feet, center of pressure of both feet, projection of anterior superior iliac spine of the pelvis (“ASIS”) and posterior superior iliac spine of the pelvis (“PSIS”) points from the pelvis on the floor, center of mass as calculated by the model, and combined center of pressure as calculated by the model.

Further, FIG. 8B depicts the application display in which the top portion of the screen 104 is a real-time top view of the two insoles with the center of pressure for each insole. Further, the bottom portion of the screen 106 shows a real-time front and side view of the full model showing the movement of the body segments of interest.

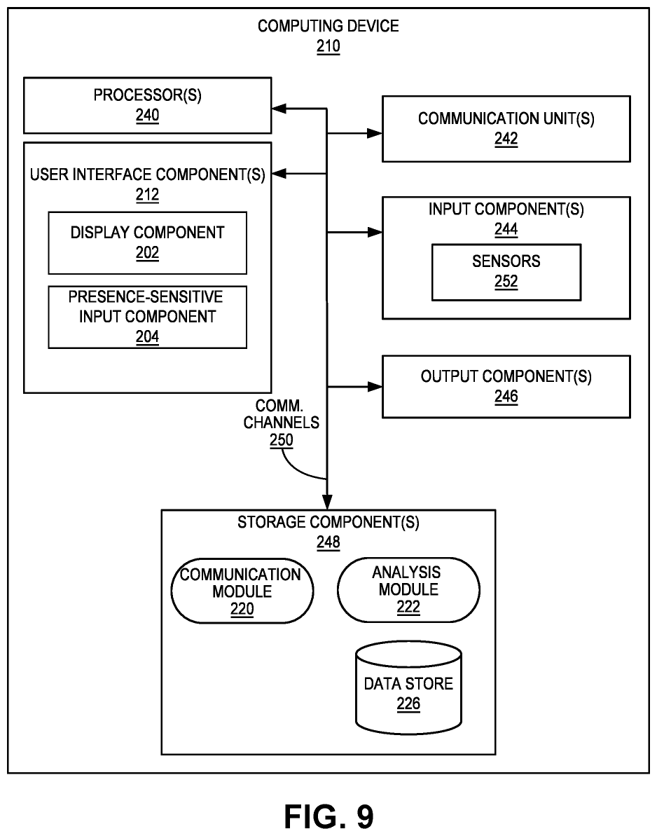

FIG. 9 is a block diagram illustrating a more detailed example of a computing device configured to perform the techniques described herein. Computing device 210 of FIG. 9 is described below as an example of a computing device that may be used in combination with or in place of the computing device 22, server 26, and network 24 discussed above and may comprise or contain central processor 18 and/or processor 20 of FIG. 1. FIG. 9 illustrates only one particular example of computing device 210, and many other examples of computing device 210 (such as device 22 and the related server(s) 26 and network 24, for example) may be used in other instances and may include a subset of the components included in example computing device 210 or may include additional components not shown in FIG. 9.

Computing device 210 may be any computer with the processing power required to adequately execute the techniques described herein. For instance, computing device 210 may be any one or more of a mobile computing device (e.g., a smartphone, a tablet computer, a laptop computer, etc.), a desktop computer, a smarthome component (e.g., a computerized appliance, a home security system, a control panel for home components, a lighting system, a smart power outlet, etc.), a vehicle, a wearable computing device (e.g., wearable sensors to provide sensory stimulation for balance, a smart watch, computerized glasses, a heart monitor, a glucose monitor, smart headphones, etc.), a virtual reality/augmented reality/extended reality (VR/AR/XR) system, a video game or streaming system, a network modem, router, or server system, or any other computerized device that may be configured to perform the techniques described herein.

As shown in the example of FIG. 9, computing device 210 includes user interface components (UIC) 212, one or more processors 240, one or more communication units 242, one or more input components 244, one or more output components 246, and one or more storage components 248. UIC 212 includes display component 202 and presence-sensitive input component 204. Storage components 248 of computing device 210 include communication module 220, analysis module 222, and data store 226.

One or more processors 240 may be similar to and/or perform similar functions as central processor 18, processor 20, the computer 22, and/or the server 26 of FIG. 1. In this way, one or more processors 240 may implement functionality and/or execute instructions associated with computing device 210 to analyze pressure sensor readings and angle sensor readings in order to provide sensory stimulation. That is, processors 240 may implement functionality and/or execute instructions associated with computing device 210 to receive and process pressure sensor and angle signals and generate and output sensory stimulation signals.

Examples of processors 240 include any combination of application processors, display controllers, auxiliary processors, one or more sensor hubs, and any other hardware configured to function as a processor, a processing unit, or a processing device, including dedicated graphical processing units (GPUs). Modules 220 and 222 may be operable by processors 240 to perform various actions, operations, or functions of computing device 210. For example, processors 240 of computing device 210 may retrieve and execute instructions stored by storage components 248 that cause processors 240 to perform the operations described with respect to modules 220 and 222. The instructions, when executed by processors 240, may cause computing device 210 to analyze pressure sensor and angle readings in order to provide sensory stimulation.

Communication module 220 may execute locally (e.g., at processors 240) to provide functions associated with receiving signals from one or more sensors (e.g., a force sensor, a pressure sensor, a motion sensor, and/or an angle sensor) and output signals to sensory stimulation units. In some examples, communication module 220 may act as an interface to a remote service accessible to computing device 210. For example, communication module 220 may be an interface or application programming interface (API) to a remote server that receives signals from one or more sensors (e.g., a force sensor, a pressure sensor, a motion sensor, and/or an angle sensor) and output signals to sensory stimulation units.

In some examples, analysis module 222 may execute locally (e.g., at processors 240) to provide functions associated with analyzing the data received by communication module 220 in order to accurately generate a patient-specific virtual biomechanical model to generate and estimated center of pressure and a center of gravity, and generate balance stimulation signals based on the estimated center of pressure and the center of gravity. In some examples, analysis module 222 may act as an interface to a remote service accessible to computing device 210. For example, analysis module 222 may be an interface or application programming interface (API) to a remote server that analyzes the data received by communication module 220 in order to accurately generate a patient-specific virtual biomechanical model to generate and estimated center of pressure and a center of gravity, and generate balance stimulation signals based on the estimated center of pressure and the center of gravity.

One or more storage components 248 within computing device 210 may store information for processing during operation of computing device 210 (e.g., computing device 210 may store data accessed by modules 220 and 222 during execution at computing device 210), including one or more patient-specific virtual biomechanical models. In some examples, storage component 248 is a temporary memory, meaning that a primary purpose of storage component 248 is not long-term storage. Storage components 248 on computing device 210 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage components 248, in some examples, also include one or more computer-readable storage media. Storage components 248 in some examples include one or more non-transitory computer-readable storage mediums. Storage components 248 may be configured to store larger amounts of information than typically stored by volatile memory. Storage components 248 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 248 may store program instructions and/or information (e.g., data) associated with modules 220 and 222 and data store 226. Storage components 248 may include a memory configured to store data or other information associated with modules 220 and 222 and data store 226.

Communication channels 250 may interconnect each of the components 212, 240, 242, 244, 246, and 248 for inter-component communications (physically, communicatively, and/or operatively). In some examples, communication channels 250 may include a system bus, a network connection, an inter-process communication data structure, or any other method for communicating data.

One or more communication units 242 of computing device 210 may communicate with external devices via one or more wired and/or wireless networks by transmitting and/or receiving network signals on one or more networks. Examples of communication units 242 include a network interface card (e.g., such as an Ethernet card), an optical transceiver, a radio frequency transceiver, a GPS receiver, a radio-frequency identification (RFID) transceiver, a near-field communication (NFC) transceiver, or any other type of device that can send and/or receive information. Other examples of communication units 242 may include short wave radios, cellular data radios, wireless network radios, as well as universal serial bus (USB) controllers.

One or more input components 244 of computing device 210 may receive input. Examples of input are tactile, audio, and video input. Input components 244 of computing device 210, in one example, include a presence-sensitive input device (e.g., a touch sensitive screen, a PSD), mouse, keyboard, voice responsive system, camera, microphone or any other type of device for detecting input from a human or machine. In some examples, input components 244 may include one or more sensor components (e.g., sensors 252). Sensors 252 may be physically incorporated into computing device 210 or may be in wired or wireless communication with computing device 210. Sensors 252 may include one or more biometric sensors (e.g., fingerprint sensors, retina scanners, vocal input sensors/microphones, facial recognition sensors, cameras), one or more location sensors (e.g., GPS components, Wi-Fi components, cellular components), one or more temperature sensors, one or more movement sensors (e.g., accelerometers, gyros), one or more pressure sensors (e.g., barometer or force sensors), one or more ambient light sensors, and one or more other sensors (e.g., infrared proximity sensor, hygrometer sensor, and the like). Other sensors, to name a few other non-limiting examples, may include a force sensor, a pressure sensor, a motion sensor, an angle sensor a radar sensor, a lidar sensor, a sonar sensor, a heart rate sensor, magnetometer, glucose sensor, olfactory sensor, compass sensor, or a step counter sensor.

One or more output components 246 of computing device 210 may generate output in a selected modality. Examples of modalities may include a tactile notification, audible notification, visual notification, machine generated voice notification, or other modalities. Output components 246 of computing device 210, in one example, include a presence-sensitive display, a sound card, a video graphics adapter card, a speaker, a cathode ray tube (CRT) monitor, a liquid crystal display (LCD), a light emitting diode (LED) display, an organic LED (OLED) display, a virtual/augmented/extended reality (VR/AR/XR) system, a three-dimensional display, or any other type of device for generating output to a human or machine in a selected modality.

UIC 212 of computing device 210 includes display component 202 and presence-sensitive input component 204. Display component 202 may be a screen, such as any of the displays or systems described with respect to output components 246, at which information (e.g., a visual indication) is displayed by UIC 212 while presence-sensitive input component 204 may detect an object at and/or near display component 202.

While illustrated as an internal component of computing device 210, UIC 212 may also represent an external component that shares a data path with computing device 210 for transmitting and/or receiving input and output. For instance, in one example, UIC 212 represents a built-in component of computing device 210 located within and physically connected to the external packaging of computing device 210 (e.g., a screen on a mobile phone). In another example, UIC 212 represents an external component of computing device 210 located outside and physically separated from the packaging or housing of computing device 210 (e.g., a monitor, a projector, etc. that shares a wired and/or wireless data path with computing device 210).

UIC 212 of computing device 210 may detect two-dimensional and/or three-dimensional gestures as input from a user of computing device 210. For instance, a sensor of UIC 212 may detect a user's movement (e.g., moving a hand, an arm, a pen, a stylus, a tactile object, etc.) within a threshold distance of the sensor of UIC 212. UIC 212 may determine a two or three-dimensional vector representation of the movement and correlate the vector representation to a gesture input (e.g., a hand-wave, a pinch, a clap, a pen stroke, etc.) that has multiple dimensions. In other words, UIC 212 can detect a multi-dimension gesture without requiring the user to gesture at or near a screen or surface at which UIC 212 outputs information for display. Instead, UIC 212 can detect a multi-dimensional gesture performed at or near a sensor which may or may not be located near the screen or surface at which UIC 212 outputs information for display.

In accordance with the techniques of this disclosure, communication module 220 may receive force and/or pressure signals including force and/or pressure information relating to the lower limb or prosthesis from at least one force and/or pressure sensor associated with at least one lower limb or prosthesis of a patient. Communication module 220 may further receive motion and/or angle signals including motion and/or angle information relating to the lower limb or prosthesis from at least one motion and/or angle sensor associated with at least one lower limb or prosthesis of a patient. Analysis module 222 may generate a patient-specific virtual biomechanical model, stored in data store 226, based on the force and/or pressure signals and the motion and/or angle signals to generate and estimated center of pressure and a center of gravity. Analysis module 222 may further generate balance stimulation signals based on the estimated center of pressure and the center of gravity. Communication module 220 may output the balance stimulation signals to at least one sensory stimulation unit disposed on at least one lower limb or prosthesis of the patient, wherein the at least one sensory stimulation unit comprises at least two stimulators that are actuable to provide stimulation to the patient based on the balance stimulation signals.

In using the techniques of this disclosure, one may more effectively assist those who suffer from conditions that affect a patient's ability to sense force, pressure, motion, or angles with their limbs. For instance, those with lower-extremity injuries or amputations or certain maladies may not be able to properly ascertain the force on certain parts of their bodies. By utilizing computing device 210 to communicate with sensors that gather force, pressure, motion, and/or angle information, analyze that information, and output sensory stimulation signals to sensory stimulation units that provide sensory stimulation to the patient in other parts of their body, patients may be more capable of walking and balancing on their own, reducing further injuries that could come from the lack of balance or feeling they may otherwise have.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A system for improving sensorimotor function of a patient, the system comprising:
(a) at least one force and/or pressure sensor associated with at least one lower limb or prosthesis of a patient, wherein the at least one force and/or pressure sensor is configured to detect force and/or pressure information relating to the lower limb or prosthesis and transmit force and/or pressure signals based on the force and/or pressure information;
(b) at least one motion and/or angle sensor associated with at least one lower limb or prosthesis of a patient, wherein the at least one motion and/or angle sensor is configured to detect motion and/or angle information relating to the lower limb or prosthesis and transmit motion and/or angle signals based on the motion and/or angle information;
(c) a processor configured to receive the force and/or pressure signals and the motion and/or angle signals, generate a patient-specific virtual biomechanical model based on the force and/or pressure signals and the motion and/or angle signals to generate an estimated center of pressure and a center of gravity, and generate balance stimulation signals based on the estimated center of pressure and the center of gravity; and
(d) at least one sensory stimulation unit disposed on at least one lower limb or prosthesis of the patient, wherein the at least one sensory stimulation unit comprises at least two stimulators that are actuable to provide stimulation to the patient based on the balance stimulation signals.

2. The system of claim 1, wherein a first of the at least one force and/or pressure sensor is associated with a first pad, wherein the first pad is disposable under a first foot or prosthetic foot of the patient.

3. The system of claim 2, wherein a second of the at least one force and/or pressure sensor is associated with a second pad, wherein the second pad is disposable under a second foot or prosthetic foot of the patient.

4. The system of claim 1, wherein the at least one motion and/or angle sensor comprises five motion and/or angle sensors.

5. The system of claim 4, wherein each of the five motion and/or angle sensors is an inertial motion unit disposed within a sensor processing module.

6. The system of claim 1, wherein the at least one sensory stimulation unit comprises a first stimulation unit disposed on a first lower limb or prosthesis of the patient and a second stimulation unit disposed on a second lower limb or prosthesis of the patient.

7. The system of claim 1, wherein the at least one sensory stimulation unit comprises four stimulators.

8. The system of claim 1, further comprising a user interface operably coupled to the processor, wherein the user interface is configured to display the patient-specific virtual biomechanical model.

9. The system of claim 8, wherein the user interface comprises an application in a mobile device.

10. The system of claim 9, wherein the mobile device comprises a laptop or a smartphone.

11. A system for improving sensorimotor function of a patient, the system comprising:
(a) at least one force and/or pressure sensor associated with at least one lower limb or prosthesis of a patient, wherein the at least one force and/or pressure sensor is configured to detect force and/or pressure information relating to the lower limb or prosthesis and transmit force and/or pressure signals based on the force and/or pressure information;
(b) at least one motion and/or angle sensor associated with at least one lower limb or prosthesis of a patient, wherein the at least one motion and/or angle sensor is configured to detect motion and/or angle information relating to the lower limb or prosthesis and transmit motion and/or angle signals based on the motion and/or angle information;
(c) a processor configured to receive the force and/or pressure signals and the motion and/or angle signals, generate a patient-specific virtual biomechanical model based on the force and/or pressure signals and the motion and/or angle signals to generate an estimated center of pressure and a center of gravity, and generate balance stimulation signals based on the estimated center of pressure and the center of gravity;
(d) at least one sensory stimulation unit disposed on at least one lower limb or prosthesis of the patient, wherein the at least one sensory stimulation unit comprises at least two stimulators that are actuable to provide stimulation to the patient based on the balance stimulation signals; and
(e) a user interface operably coupled to the processor, wherein the user interface is configured to receive information from the processor about the patient-specific virtual biomechanical model and display the patient-specific virtual biomechanical model based on the information from the processor.

12. The system of claim 11, wherein a first of the at least one force and/or pressure sensor is associated with a first pad, wherein the first pad is disposable under a first foot or prosthetic foot of the patient and a second of the at least one force and/or pressure sensor is associated with a second pad, wherein the second pad is disposable under a second foot or prosthetic foot of the patient.

13. The system of claim 11, wherein the at least one motion and/or angle sensor comprises five motion and/or angle sensors, wherein first and second motion and/or angle sensors are disposed on a first lower limb or prosthesis of the patient, third and fourth motion and/or angle sensors are disposed on a second lower limb or prosthesis of the patient, and a fifth motion and/or angle sensor is disposed on a lower back of the patient.

14. The system of claim 13, wherein each of the five motion and/or angle sensors is an inertial motion unit disposed within a sensor processing module, wherein the fifth motion and/or angle sensor is operably coupled to a local central processor, wherein the local central processor is in communication with the processor.

15. The system of claim 11, wherein the at least one sensory stimulation unit comprises a first stimulation unit disposed on a first lower limb or prosthesis of the patient and a second stimulation unit disposed on a second lower limb or prosthesis of the patient, wherein each of the first and second stimulation units comprises:
(a) a band configured to be couplable to a lower limb or prosthesis;
(b) the at least two stimulators comprising four stimulators attached to the band; and
(c) one of the at least one motion and/or angle sensor associated with one of the four stimulators.

16. The system of claim 11, wherein the user interface comprises an application in a mobile device, wherein the mobile device comprises a laptop, or a smartphone.

17. A system for improving sensorimotor function of a patient, the system comprising:
(a) a first footpad unit comprising a first footpad comprising at least one first force and/or pressure sensor positionable under a first foot or prosthetic foot of a first lower limb or prosthesis of a patient, and a second footpad unit comprising a second footpad comprising at least one second force and/or pressure sensor positionable under a second foot or prosthetic foot of a second lower limb or prosthesis of the patient, wherein each of the at least one first and second force and/or pressure sensors are configured to detect force and/or pressure information relating to the first and second lower limbs or prostheses, respectively, and transmit force and/or pressure signals based on the force and/or pressure information;
(b) first and second sensor processing modules comprising at least one first motion and/or angle sensor associated with the first lower limb or prosthesis of the patient, third and fourth sensor processing modules comprising at least one second motion and/or angle sensor associated with the second lower limb or prosthesis of the patient, and a fifth sensor processing module comprising at least one third motion and/or angle sensor associated with a lower back of the patient, wherein each of the at least one first, second, and third motion and/or angle sensors is configured to detect motion and/or angle information and transmit motion and/or angle signals based on the motion and/or angle information;
(c) a processor configured to receive the force and/or pressure signals and the motion and/or angle signals, generate a patient-specific virtual biomechanical model based on the force and/or pressure signals and the motion and/or angle signals to generate an estimated center of pressure and a center of gravity, and generate balance stimulation signals based on the estimated center of pressure and the center of gravity;
(d) at least one sensory stimulation unit disposed on at least one lower limb or prosthesis of the patient, wherein the at least one sensory stimulation unit comprises at least two stimulators that are actuable to provide stimulation to the patient based on the balance stimulation signals; and
(e) a user interface operably coupled to the processor, wherein the user interface is configured to receive information from the processor about the patient-specific virtual biomechanical model and display the patient-specific virtual biomechanical model based on the information from the processor.

18. The system of claim 17, wherein the fifth sensor processing module comprises a local central processor, wherein the local central processor is in communication with the processor.

19. The system of claim 17, wherein the at least one sensory stimulation unit comprises:
(a) a first stimulation unit disposed on the first lower limb or prosthesis of the patient, the first stimulation unit comprising:
(i) a first band configured to be couplable to the first lower limb or prosthesis;
(ii) four first stimulators attached to the first band; and
(iii) one of the first and second sensor processing modules associated with one of the four stimulators; and
(b) a second stimulation unit disposed on the second lower limb or prosthesis of the patient, the second stimulation unit comprising:
(i) a second band configured to be couplable to the second lower limb or prosthesis;
(ii) four second stimulators attached to the second band; and
(iii) one of the third and fourth sensor processing modules associated with one of the four stimulators.

20. The system of claim 17, wherein the user interface comprises an application in a mobile device, wherein the mobile device comprises a laptop or a smartphone.
